(19) United States
(12) Patent Application Publication
(10) Pub. No.: US 2023/0047024 A1
(43) Pub. Date: Feb. 16, 2023

(54) TROCAR ASSEMBLIES AND TUNNELING SYSTEMS INCLUDING TROCAR ASSEMBLIES

(71) Applicant: InCube Labs, LLC, San Jose, CA (US)

(72) Inventors: Kyle HORLEN, Shavano Park, TX (US); Charles Gregory NELSON, New Braunfels, TX (US)

(21) Appl. No.: 17/873,419

(22) Filed: Jul. 26, 2022

Related U.S. Application Data
(60) Provisional application No. 63/231,965, filed on Aug. 11, 2021.

(51) Int. Cl. A61B 17/34; A61N 1/05

(52) U.S. Cl. CPC A61B 17/3417; A61N 1/0551; A61B 17/3468

(57) ABSTRACT

A trocar assembly comprises an elongate shaft, a piercing tip at a distal end of the shaft, a handle at a proximal end of the shaft, and a retaining member. When a passer tube is positioned over the shaft, the retaining member engages with an inner surface or an outer surface of the passer tube to retain a position of the passer tube over the shaft.

### CROSS-REFERENCE TO RELATED APPLICATIONS

This application claims the benefit of U.S. provisional application Ser. No. 63/231,965 filed Aug. 11, 2021, the disclosure of which is hereby incorporated in its entirety by reference herein.

### TECHNICAL FIELD

Embodiments relate to trocar assemblies and tunneling systems including trocar assemblies.

### BACKGROUND

A medical device may be implanted within a recipient at an implant location that is at a distance from a target location (e.g., a treatment location) within the recipient. The medical device may be physically and/or electrically connected to the target location, or to another medical device located at the target location, by using a tunneling system to establish this connection. For example, an implantable pulse generator (IPG) may be implanted within a recipient and an electrode lead may electrically connect the IPG to an electrode positioned adjacent to or surrounding a nerve at a distant target nerve location to provide neurostimulation to the nerve. The electrode lead may be tunneled through the recipient from the IPG implantation site to the target nerve location using a tunneling system.

A conventional tunneling system includes a passer tube and a trocar having a removable piercing tip at a distal end of a shaft. The piercing tip has a maximum diameter larger than an inside diameter of the passer tube so that, when the passer tube is positioned over the shaft, the distal end of the passer tube encounters the piercing tip, thus preventing the passer tube from falling off the shaft. After the piercing tip and passer tube have been tunneled to a target location, the piercing tip must be removed from the shaft (such as by unscrewing the piercing tip) so the shaft can be pulled back through the passer tube and an electrode lead (or other connector) can be passed through the passer tube. However, removing the small piercing tip can be dangerous to the recipient and surgeon as the sharp point may injure the surgeon and/or the recipient and may cut gloves, thereby jeopardizing the sterility of the surgical environment.

An alternate tunneling system includes a passer tube mounted to a handle and a trocar having a piercing tip at a distal end of a shaft. The piercing tip has a maximum diameter larger than an inside diameter of the passer tube so that, when the passer tube is positioned over the shaft, the distal end of the passer tube encounters the piercing tip, thus preventing the passer tube from falling off the shaft. After tunneling to a target location, the trocar (piercing tip and shaft) is removed from the passer tube in the same direction of tunneling, while the passer tube and handle remain in place within the recipient. A lead (or other connector) may then be passed through the handle and passer tube to connect the implantation location and the target location. However, the large size of the handle requires that the tunneling system be introduced only from the implantation site (e.g., IPG implantation site) and pushed toward the target location (e.g., target nerve location). This may result in damaging tissue (e.g., nerve tissue) at the target location. Furthermore, the large size of the handle requires that the passer tube be removed from the recipient only in the direction opposite to the direction of tunneling.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 shows an illustrative tunneling system including a trocar assembly and a passer tube that may be used during a surgical procedure to perform a tunneling operation.

FIG. 2 shows the tunneling system of FIG. 1 when the passer tube is positioned on the trocar assembly.

FIGS. 3A and 3B show cross-sectional views of an illustrative configuration of the proximal end of the trocar assembly of FIGS. 1 and 2.

FIGS. 4A and 4B show cross-sectional views of another illustrative configuration of the proximal end of the trocar assembly of FIGS. 1 and 2.

FIGS. 5A and 5B show cross-sectional views of another illustrative configuration of the proximal end of the trocar assembly of FIGS. 1 and 2.

FIGS. 6A and 6B show cross-sectional views of another illustrative configuration of the proximal end of the trocar assembly of FIGS. 1 and 2.

FIGS. 7A-12 show different stages of an illustrative surgical procedure performed with the tunneling system and trocar assembly of FIGS. 1 and 2.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

Trocar assemblies and tunneling systems, and methods of using trocar assemblies and tunneling systems, are described herein. For example, an illustrative trocar assembly may include an elongate shaft, a piercing tip at a distal end of the shaft, a handle at a proximal end of the shaft, and a retaining member. When a passer tube is positioned over the shaft, the retaining member engages with an inner surface or an outer surface of the passer tube to retain a position of the passer tube over the shaft. The retaining member may include, for example, an O-ring on the shaft or in the handle or a seal member (e.g., a lip seal or a cup seal) in the handle. An illustrative tunneling system may include the passer tube and the trocar assembly.

The trocar assembly and tunneling system may be used for establishing a path for connecting (e.g., physically and/or electrically) devices and/or anatomical features at different locations within a recipient, such as connecting a device and/or anatomical feature at a first site within the recipient with a device and/or anatomical feature at a second site within the recipient. For example, the trocar assembly and tunneling system may be used to position an electrode lead to electrically connect an IPG implanted at the first site to an electrode adjacent to or surrounding a nerve at the second site.

As used herein, a “recipient” may include a body of a live human or animal, a human or animal cadaver, a portion of human or animal anatomy, tissue removed from human or animal anatomies, non-tissue work pieces, a training model, a dummy, etc.

To illustrate, the trocar assembly may be inserted into a proximal end of the passer tube and pushed through the passer tube so that the piercing tip emerges from a distal end of the passer tube and the passer tube is positioned over the shaft of the trocar assembly. While the passer tube is positioned over the shaft, the piercing tip may be pushed from the first site (e.g., the IPG implantation site) to the second site (e.g., the nerve site), or vice versa. The trocar assembly may then be removed from the passer tube by pulling the trocar assembly back through the passer tube so that the shaft and the piercing tip emerge from the proximal end of the passer tube. The passer tube may then be used as a channel between the first site and the second site.

For example, an electrode lead may be passed through the passer tube between the first site (e.g., the IPG implantation site) and the second site (e.g., the nerve site). The passer tube may then be removed from the recipient while leaving the electrode lead in place extending from the first site to the second site. In an embodiment with an IPG to be electrically connected to one or more electrode(s) at a nerve site by way of an electrode lead, a proximal end of the electrode lead may be connected to the IPG at the IPG implantation site (e.g., the first site) and the distal end of the electrode lead may be positioned adjacent to the nerve at the nerve site (such that electrodes incorporated at the distal end of the electrode lead may be positioned adjacent to or around the nerve) or may be connected to a separate electrode or electrode assembly which is positioned at the nerve site. The electrode lead may be connected at the proximal or distal end prior to or after being passed through the passer tube.

The trocar assemblies, tunneling systems, and methods described herein may provide various benefits and advantages over conventional trocar assemblies, tunneling systems, and methods. For example, the trocar assemblies described herein securely retain (such as by friction) the passer tube over the shaft so the passer tube does not fall off the shaft before being inserted into a recipient while allowing easy removal of the trocar assembly from the passer tube by the simple application of a little force greater than the retaining force. The trocar assemblies are also safer and more convenient than conventional trocar assemblies because the piercing tip does not need to be removed from the shaft to remove the trocar assembly from the passer tube.

The trocar assemblies and tunneling systems described herein may be inserted into the recipient at any desired location, such as at an IPG implantation site or at a nerve site or at an electrode implantation site. Moreover, a trocar assembly may be removed from the passer tube in the opposite direction of insertion of the trocar assembly and tunneling system into the recipient, thus potentially simplifying surgical procedures. In some embodiments, a tunneling system may include a passer tube that is large enough to allow multiple electrode leads with connectors to fit within the passer tube concurrently during passing of the electrode leads through the passer tube or during removal of the passer tube over the electrode leads. This may obviate the need to pass the tunneling system through tissue a second time, thereby reducing injury to the recipient as well as shortening duration of the surgical procedure.

For at least these reasons, the trocar assemblies and tunneling systems described herein enable safer, faster, easier, and more convenient surgical procedures and reduce patient time and staff time in surgery, as compared with conventional trocar assemblies and tunneling systems.

Various embodiments will now be described in more detail with reference to the figures. The systems, assemblies, and methods described herein may provide one or more of the benefits mentioned above and/or various additional and/or alternative benefits that will be made apparent herein.

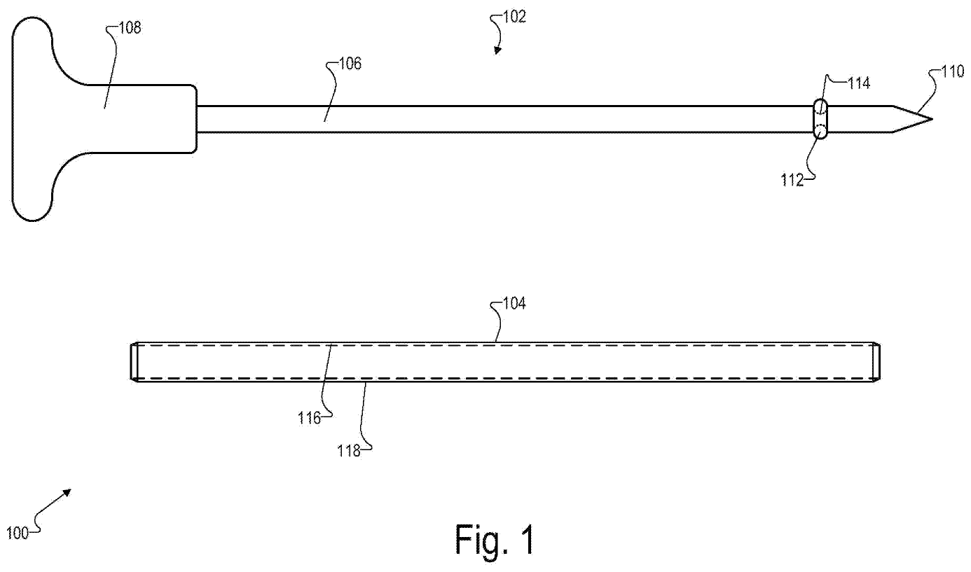

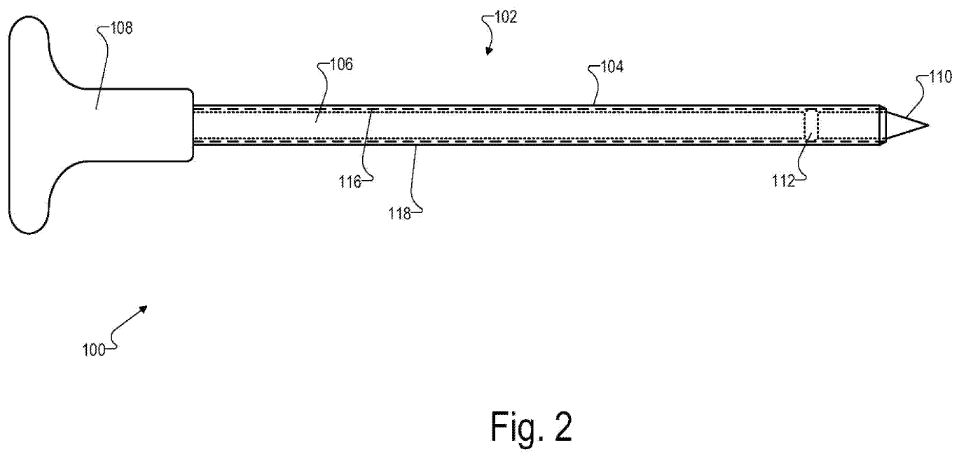

FIGS. 1 and 2 show an illustrative tunneling system 100 that may be used during a surgical procedure to perform a tunneling operation. As shown, tunneling system 100 includes a trocar assembly 102 and a passer tube 104 configured to be positioned on trocar assembly 102. FIG. 1 shows trocar assembly 102 separated from passer tube 104, and FIG. 2 shows tunneling system 100 when passer tube 104 is positioned on trocar assembly 102. As will be explained below in more detail, tunneling system 100 may be inserted into a recipient and a distal end of tunneling system 100 may be pushed to a target location within the recipient. Trocar assembly 102 may be removed from passer tube 104 while passer tube 104 remains within the recipient. A medical device (e.g., an electrode lead) may then be inserted through passer tube 104 and pushed to the target location. Passer tube 104 may then be removed from the recipient. Thus, tunneling system 100 may be used to tunnel a medical device to the target location within the recipient.

As shown in FIGS. 1 and 2, trocar assembly 102 includes a shaft 106, a piercing tip 110 at a distal end of shaft 106, a handle 108 at a proximal end of shaft 106, and an O-ring 112 as a retaining member configured to retain a position of passer tube 104 on trocar assembly 102. As used herein, “proximal” refers to a position near handle 108 and “distal” refers to a position away from handle 108.

Shaft 106 is an elongate rod and may have any suitable cross-sectional shape (e.g., circular, rectangular, square, triangular, elliptical, etc.) and size and may be made of any suitable material, such as metal (e.g., titanium, stainless steel, etc.), a polymer, or a composite material.

Handle 108 is positioned at a proximal end of shaft 106 and is secured to shaft 106. Handle 108 may be secured to shaft 106 in any suitable way, such as by an adhesive, by a threaded connection, by a fastener, by friction, by a mechanical connection (e.g., snap or clip connection), etc. Alternatively, handle 108 may be formed integrally with shaft 106. A user may grasp handle 108 to push a distal end of tunneling system 100 into the recipient and to remove trocar assembly 102 from passer tube 104. Handle 108 may have any suitable shape and configuration. For example, as shown in FIGS. 1 and 2 handle 108 has a “T” shape. In other examples, handle 108 may have a spherical shape, a “knob” shape, or any other suitable shape.

Piercing tip 110 is located at a distal end of shaft 106 and is configured to cut through the recipient (e.g., tissue) as tunneling system 100 is pushed into and through the recipient toward a target location. Piercing tip 110 may have any suitable shape and profile (e.g., conical). In some embodiments, piercing tip 110 is formed integrally with shaft 106 (e.g., shaft 106 and piercing tip 110 form a unitary body and thus need not be joined by any physical or mechanical connection). In alternative embodiments, piercing tip 110 is formed separately from shaft 106 and is attached to shaft 106, such as by a threaded connection or other physical or mechanical connection.

O-ring 112 is configured to retain, by friction, a position of passer tube 104 over shaft 106. As shown in FIG. 1, O-ring 112 is positioned in a groove 114 on shaft 106 to maintain a position of O-ring 112 on shaft 106. O-ring 112 and groove 114 may have any suitable cross-sectional profiles. For example, O-ring 112 may have a rectangular or square cross-sectional profile to prevent O-ring 112 from rolling when shaft 106 is inserted in and removed from passer tube 104.

O-ring 112 has an outside diameter that is slightly larger than the inside diameter of passer tube 104 to thereby create friction with an inner surface 116 of passer tube 104. The positioning of O-ring 112 in groove 114 retains O-ring 122 in position and thus can assist in maintaining the outside diameter of O-ring 112 to create the friction. O-ring 112 may be formed of any suitable material configured to create friction with inner surface 116 of passer tube 104. For example, O-ring 112 may be formed of an elastomeric material such as silicone rubber, natural rubber, a fluoroelastomer (such as polytetrafluoroethylene (PTFE), perfluoroether (PFA), fluorinated ethylene propylene (FEP)), ethylene propylene diene monomer (EPDM), nitrile rubber (e.g., acrylonitrile-butadiene rubber), a polyolefin elastomer, polyurethane, and/or any other suitable elastomer. In some examples, O-ring 112 may have a Shore A hardness ranging from about 50 to about 80 durometer. In further examples, O-ring 112 may have a Shore A hardness ranging from about 60 to about 75 durometer. In yet further examples, O-ring 112 may have a Shore A hardness ranging from about 65 to about 75 durometer. In some examples, O-ring 112 is formed of EPDM and has a Shore A hardness of about 70 durometer, which enables O-ring 112 to withstand autoclave temperatures.

FIG. 1 shows that O-ring 112 and groove 114 are positioned at the distal end of shaft 106 near piercing tip 110. However, O-ring 112 and groove 114 may be positioned at any other suitable location along shaft 106, such as at a middle portion of shaft 106 or at a proximal end of shaft 106.

Passer tube 104 is an elongate, hollow tube configured to be removably positioned over shaft 106 and includes inner surface 116 and an outer surface 118. To position passer tube 104 on trocar assembly 102 over shaft 106, piercing tip 110 and shaft 106 may be inserted into a proximal end of passer tube 104 and pushed through passer tube 104 so that piercing tip 110 emerges from a distal end of passer tube 104. When passer tube 104 is positioned over shaft 106, as shown in FIG. 2, O-ring 112 engages with inner surface 116 of passer tube 104 to retain, by friction, a position of passer tube 104 over shaft 106 so that passer tube 104 does not fall off or slide down shaft 106. Trocar assembly 102 may then be removed from passer tube 104 by simply pulling handle 108 with a force greater than the retaining force (e.g., friction force) of O-ring 112 to pull shaft 106 and piercing tip 110 back through passer tube 104 so that shaft 106 and piercing tip 110 emerge from the proximal end of passer tube 104.

To enable movement of piercing tip 110 through passer tube 104, a maximum diameter of piercing tip 110 is less than an inside diameter of passer tube 104. With this configuration, trocar assembly 102 may be removed from passer tube 104 in the opposite direction in which trocar assembly 102 was inserted into passer tube 104. In some examples, the maximum diameter of piercing tip 110 is less than or equal to a diameter of shaft 106.

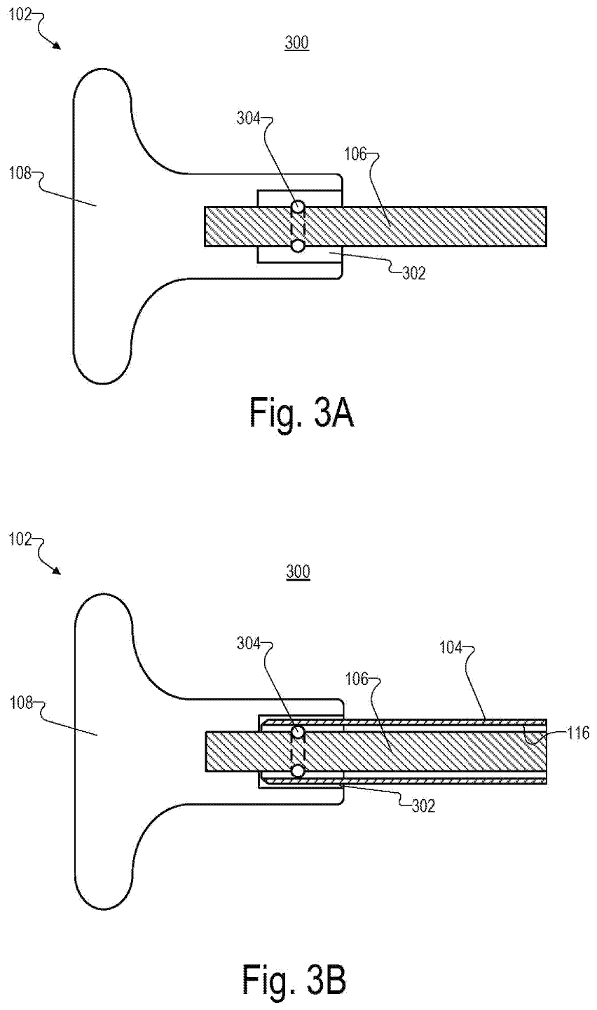

In the examples of FIGS. 1 and 2, trocar assembly 102 includes O-ring 112 as a retaining member configured to retain a position of passer tube 104 over shaft 106. In additional or alternative examples, a retaining member may be included in handle 108, as will now be shown and described with reference to FIGS. 3A-6B.

FIGS. 3A and 3B show an illustrative configuration 300 of the proximal end portion of trocar assembly 102. FIG. 3A shows a cross-sectional view of the proximal end portion of trocar assembly 102 and FIG. 3B shows a cross-sectional view of the proximal end portion of trocar assembly 102 when passer tube 104 is positioned over shaft 106. As shown in FIGS. 3A and 3B, handle 108 includes a hollow channel 302 into which the proximal end of shaft 106 is inserted for attachment to handle 108. Channel 302 is also configured to receive a proximal end of passer tube 104 when passer tube 104 is positioned over shaft 106. To this end, channel 302 has a diameter slightly larger than the outside diameter of passer tube 104. The proximal end of shaft 106 includes an O-ring 304 positioned in a groove (not shown) on the proximal end of shaft 106. However, the groove may be omitted if O-ring 304 is secured to shaft 106 by some other means (e.g., an adhesive). O-ring 304 may be similar in construction to O-ring 112 and functions as a retaining member by engaging with inner surface 116 of passer tube 104 to retain, by friction, the position of passer tube 104 on shaft 106. O-ring 304 has an outside diameter that is slightly larger than the inside diameter of passer tube 104 to thereby create friction with inner surface 116.

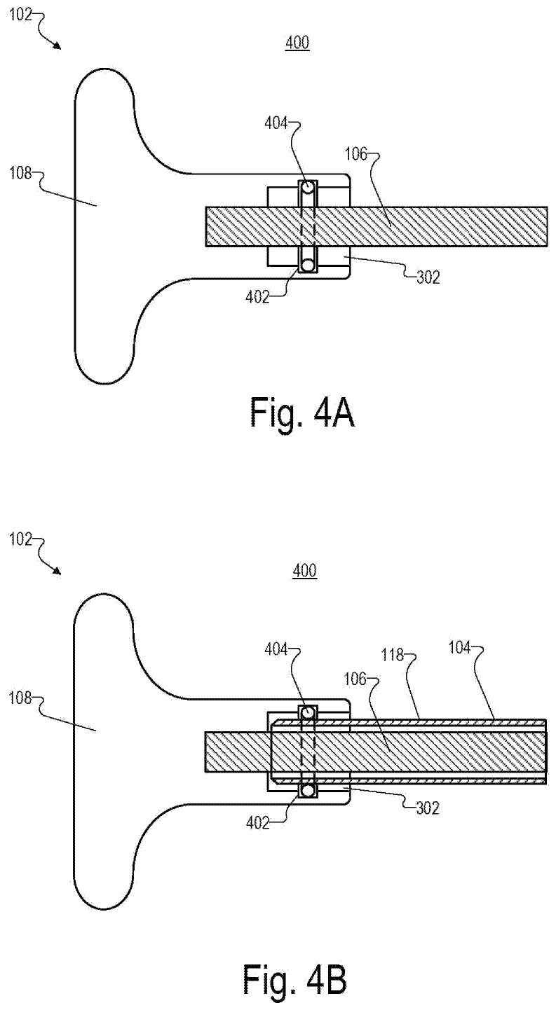

FIGS. 4A and 4B show another illustrative configuration 400 of the proximal end portion of trocar assembly 102. FIG. 4A shows a cross-sectional view of the proximal end portion of trocar assembly 102 and FIG. 4B shows a cross-sectional view of the proximal end portion of trocar assembly 102 when passer tube 104 is positioned over shaft 106. Configuration 400 is similar to configuration 300 except that, in configuration 400, a groove 402 is formed in an inside surface of channel 302 instead of on shaft 106 and an O-ring 404 is positioned in groove 402. O-ring 404 may be similar in construction to O-ring 112 and functions as a retaining member by engaging with outer surface 118 of passer tube 104 to retain, by friction, the position of passer tube 104 over shaft 106. O-ring 404 has an inside diameter that is slightly smaller than the outside diameter of passer tube 104 to thereby create friction with outer surface 118.

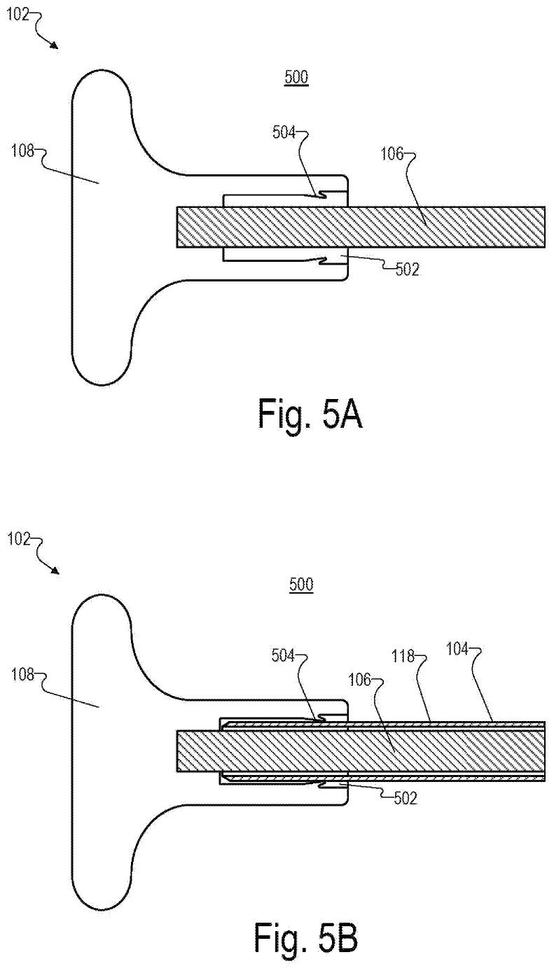

FIGS. 5A and 5B show another illustrative configuration 500 of the proximal end of trocar assembly 102. FIG. 5A shows a cross-sectional view of the proximal end portion of trocar assembly 102 and FIG. 5B shows a cross-sectional view of the proximal end portion of trocar assembly 102 when passer tube 104 is positioned over shaft 106. As shown in FIGS. 5A and 5B, handle 108 includes a hollow channel 502 into which the proximal end of shaft 106 is inserted for attachment to handle 108. Channel 502 is also configured to receive a proximal end of passer tube 104 when passer tube 104 is positioned over shaft 106. To this end, channel 502 has a diameter slightly larger than the outside diameter of passer tube 104.

A seal member 504 is formed on or attached to an inner surface of channel 502. Seal member 504 may be formed of any suitable elastomeric material described herein and functions as a retaining member by engaging with outer surface 118 of passer tube 104 to retain, by friction, the position of passer tube 104 on shaft 106. Seal member 504 has an inside diameter that is slightly smaller than the outside diameter of passer tube 104 to thereby create friction with outer surface 118. Seal member 504 may be any suitable type of seal and may have any suitable shape and profile. For example, seal member 504 may be a lip seal, a cup seal, or any other suitable friction-forming seal. It will be understood that seal member 504 need not actually perform a fluid sealing function to function as a retaining member.

Seal member 504 may be formed integrally with handle 108. For example, handle 108 may be formed in a one-shot molding process by molding handle 108 over a core pin that is shaped to form seal member 504 in handle 108. Handle 108 may alternatively be formed by additive manufacturing (e.g., 3D printing) or any other suitable process.

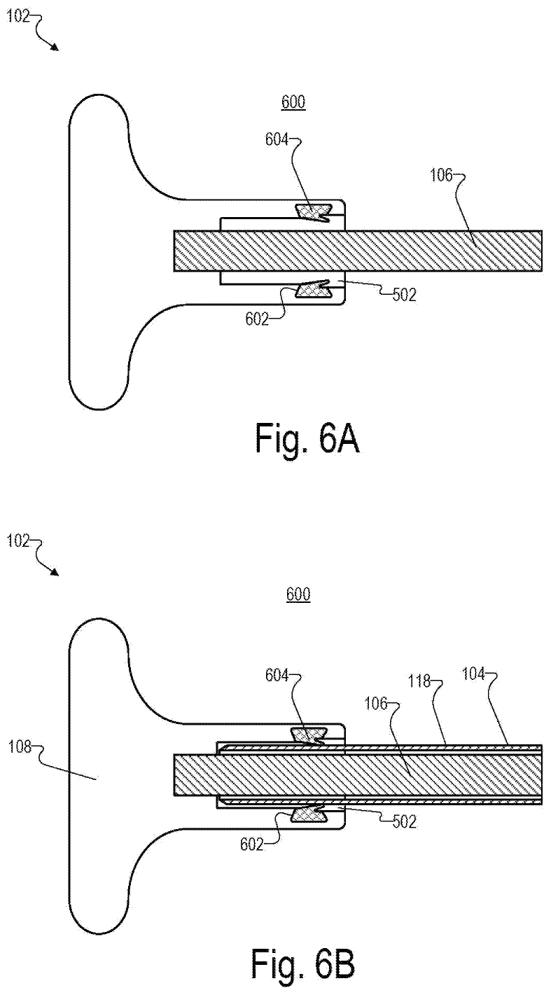

FIGS. 6A and 6B show another illustrative configuration 600 of the proximal end of trocar assembly 102. FIG. 6A shows a cross-sectional view of the proximal end portion of trocar assembly 102 and FIG. 6B shows a cross-sectional view of the proximal end portion of trocar assembly 102 when passer tube 104 is positioned over shaft 106. Configuration 600 is similar to configuration 500 except that, in configuration 600, channel 502 includes a groove 602 and a separately formed seal member 604 positioned within groove 602 and projecting from the inner surface of channel 502. Seal member 604 may be formed of any suitable elastomeric material described herein and functions as a retaining member by engaging with outer surface 118 of passer tube 104 to retain, by friction, the position of passer tube 104 on shaft 106. Seal member 604 has an inside diameter that is slightly smaller than the outside diameter of passer tube 104 to thereby create friction with outer surface 118. Seal member 604 may be any suitable type of seal and may have any suitable shape and profile. For example, seal member 604 may be a lip seal, a cup seal, or any other suitable friction-forming seal member. It will be understood that seal member 604 need not actually perform a fluid sealing function to function as a retaining member.

In some examples, handle 108 shown in FIGS. 6A and 6B is formed in a two-shot molding process. In the first shot, a core pin is placed in a seal mold and seal member 604 is molded over the core pin. In the second shot, the core pin with molded seal member 604 is placed in a handle mold and handle 108 is overmolded on seal member 604. The core pin may then be removed, leaving seal member 604 within groove 602 of handle 108. In the examples shown in FIGS. 6A and 6B, seal member 604 includes undercuts (e.g., flared outer sides) to prevent seal member 604 from falling out of groove 602. Alternatively to the two-shot molding process, handle 108 (or any of its components) may be formed by additive manufacturing (e.g., 3D printing) or any other suitable process.

It will be recognized that various modifications may be made to the embodiments described above. For example, trocar assembly 102 may include multiple retaining members in any suitable combination and configuration. For instance, trocar assembly 102 may include a first O-ring (e.g., O-ring 112) positioned at the distal end of shaft 106 and a second O-ring (e.g., O-ring 304) positioned at the proximal end of shaft 106 within handle 108. In other examples, the second O-ring (e.g., O-ring 304) is positioned on shaft 106 outside of handle 108. In further examples, the second O-ring (e.g., O-ring 404) is positioned within handle 108 rather than on shaft 106. In yet further examples, multiple O-rings are positioned in handle 108, such as O-ring 304 in a groove on shaft 106 and O-ring 404 in groove 402 in channel 302. In further examples, trocar assembly 102 includes an O-ring (e.g., O-ring 112) positioned on shaft 106 (e.g., at a distal end of shaft 106) and a seal member (e.g., seal member 504 or seal member 604) within handle 108. In other examples, trocar assembly 102 may include only one retaining member (e.g., O-ring 112, O-ring 304, O-ring 404, seal member 504, or seal member 604). It is to be understood that a retaining member (e.g., O-ring or seal member) may perform both a sealing function and a retention function. In some examples, the retaining member allows fluid to pass; in other examples, the retaining member blocks passage of fluids. In some examples, trocar assembly 102 fits snugly within passer tube 104 to prevent tissue (e.g., fat) from entering the passer tube; in such an embodiment, fluid may be allowed to pass or may be blocked.

In some examples, trocar assembly 102 may include one or more retaining members other than an O-ring or a seal member. For example, a retaining member may be implemented by any suitable pressing member(s) configured to press against inner surface 116 and/or outer surface 118 of passer tube 104. For instance, one or more elastomeric studs may be embedded in or attached to shaft 106 and protrude from the surface of shaft 106 to engage with inner surface 116 of passer tube 104. Additionally or alternatively, one or more elastomeric studs may be embedded in or attached to the inner surface of channel 302 or channel 502 and protrude from the inner surface of channel 302 or channel 502 to engage with outer surface 118 of passer tube 104.

In further examples, a retaining member may be implemented by an elastomeric sleeve that may be positioned over shaft 106. The sleeve may function similarly to an O-ring or seal member in that the sleeve may engage with inner surface 116 of passer tube to retain, by friction, the position of passer tube 104 over shaft 106. In further examples, a retaining member may be implemented by an elastomeric sleeve positioned in channel 302 or channel 502 and into which passer tube 104 may be inserted. In this example, the sleeve may have an inside diameter that is slightly smaller than the outside diameter of passer tube 104 and/or may have a seal member (e.g., seal member 504 or seal member 604) formed on an inner surface of the sleeve. In yet further examples, a retaining member may be implemented by one or more pressing members on shaft 106 and/or within handle 108 and mechanically biased (e.g., by a spring) toward passer tube 104 to engage with (e.g., press against) inner surface 116 or outer surface 118 of passer tube 104.

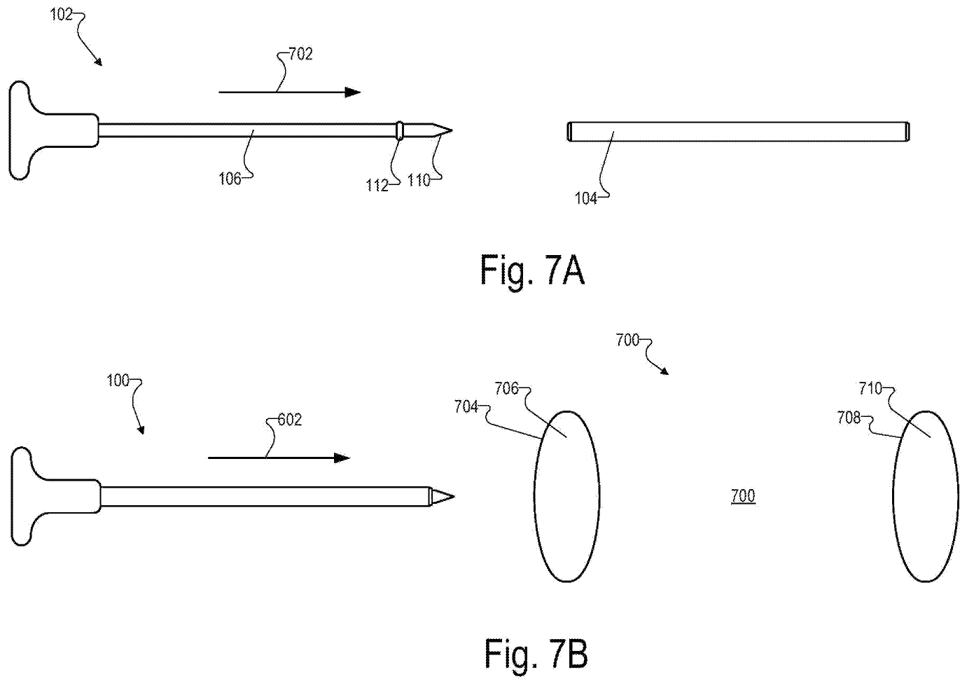

An illustrative procedure performed on a recipient 700 and using tunneling system 100 and trocar assembly 102 will now be described with reference to FIGS. 7-11. It will be recognized that the following procedure is merely illustrative of the various different procedures that may be performed with tunneling system 100 and trocar assembly 102, and that other procedures may be performed using tunneling system 100 and trocar assembly 102. Moreover, while FIGS. 7-11 show a configuration of trocar assembly 102 having an O-ring at a distal end portion of shaft 106, any configuration of trocar assembly 102 and retaining members described herein may be used.

As shown in FIG. 7A, passer tube 104 is positioned on trocar assembly 102 over shaft 106 by inserting piercing tip 110 and shaft 106 into a proximal end of passer tube 104 and pushing piercing tip 110 and shaft 106, as indicated by arrow 702, so that piercing tip 110 emerges from a distal end of passer tube 104. When passer tube 104 is positioned over shaft 106, as shown in FIG. 7B, O-ring 112 (not shown in FIG. 7B) engages with inner surface 116 of passer tube 104 to retain, by friction, a position of passer tube 104 over shaft 106 so that passer tube 104 does not fall off or slide down shaft 106.

Referring now to FIG. 7B, a first opening 704 (e.g., an incision through skin) may be formed in recipient 700 to expose a first location 706 within recipient 700 and a second opening 708 (e.g., another incision through skin) may be formed in recipient 700 to expose a second location 710 within recipient 700. First location 706 or second location 710 may be configured for implantation of a first implantable medical device during the surgical procedure, and the other of the first location 706 or second location 710 may be configured for implantation of a second medical device during the surgical procedure.

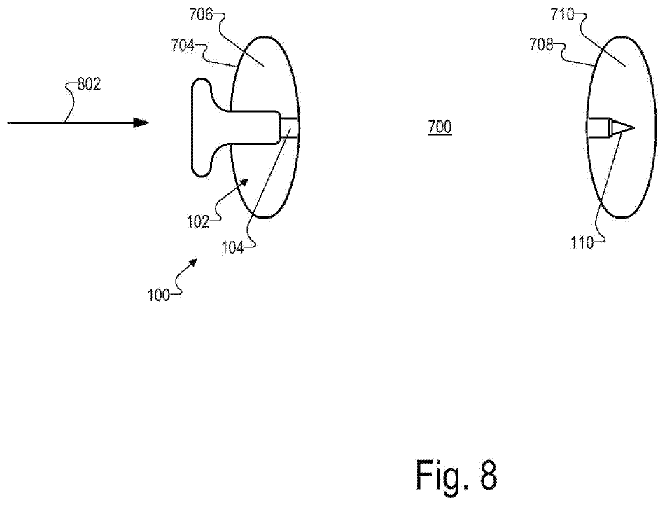

Referring now to FIG. 8, to facilitate connection of the first medical device and the second medical device, tunneling system 100 (e.g., trocar assembly 102 with passer tube 104 positioned over shaft 106 (not visible in FIG. 8)) may be inserted through first opening 704 and pushed, while passer tube 104 is positioned over shaft 106, to push piercing tip 110 and the distal end of passer tube 104 from first location 706 to second location 710, as indicated by arrow 802.

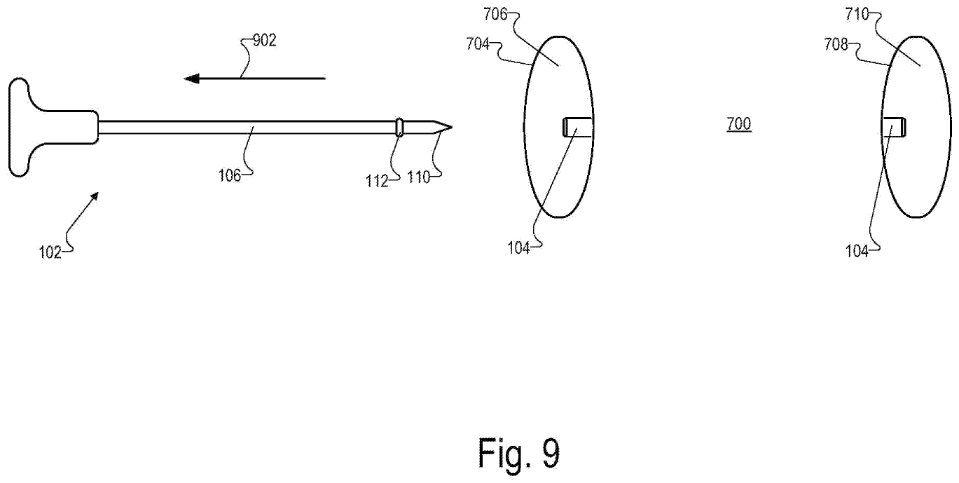

Once piercing tip 110 and the distal end of passer tube 104 are located at second location 710, trocar assembly 102 may be removed from passer tube 104, as shown in FIG. 9. Trocar assembly 102 may be removed by pulling trocar assembly 102 with a removing force greater than the retaining force of O-ring 112 so that shaft 106 and piercing tip 110 are pulled back through passer tube 104 and emerge from the proximal end of the passer tube 104, thus leaving passer tube 104 within recipient 700. Thus, trocar assembly 102 is removed from passer tube 104 in the opposite direction in which trocar assembly 102 was inserted into passer tube 104, as indicated by arrow 902. By using trocar assembly 102, piercing tip 110 does not need to be removed from shaft 106 to remove trocar assembly 102 from passer tube 104.

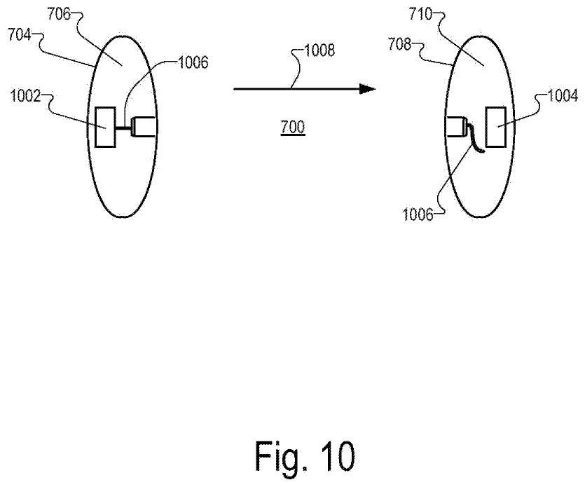

Referring now to FIG. 10, a first medical device 1002 may be implanted at first location 706 and a second medical device 1004 may be implanted at second location 710. To connect first medical device 1002 and second medical device 1004, a connector 1006 may be inserted and passed through passer tube 104 from first location 706 and/or first medical device 1002 to second location 710 and/or second medical device 1004, in the direction indicated by arrow 1008. Alternatively, connector 1006 may be inserted and passed through passer tube 104 from second location 710 and/or second medical device 1004 to first location 706 and/or first medical device 1002.

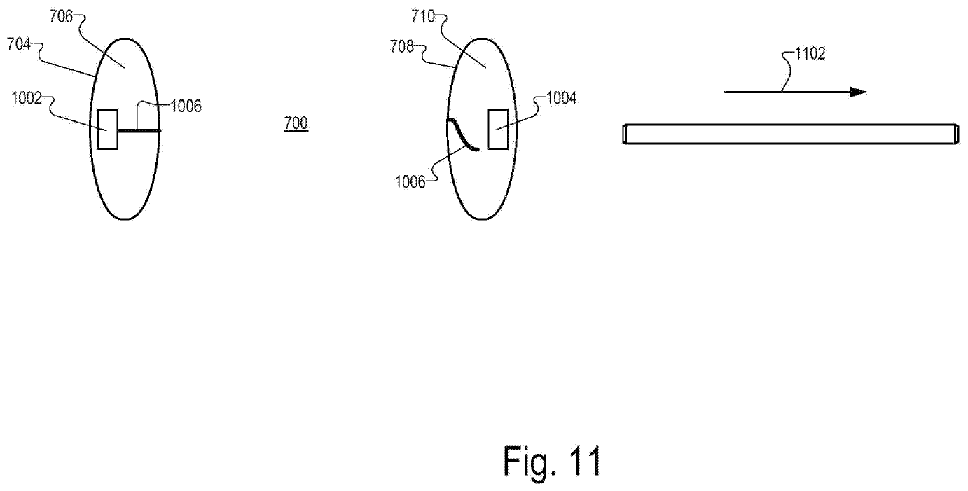

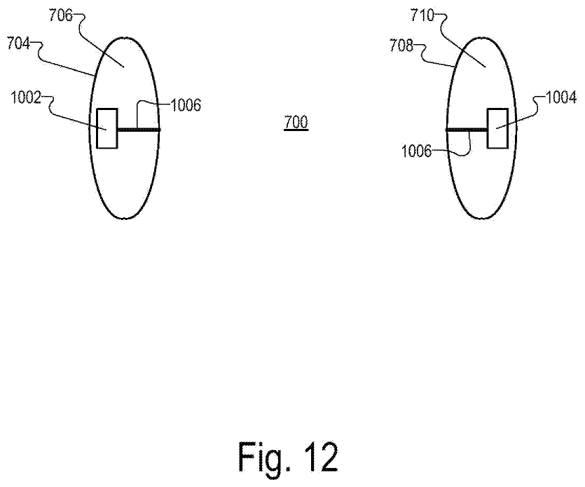

Referring now to FIG. 11, prior to connecting connector 1006 to second medical device 1004 (or first medical device 1002), passer tube 104 may be removed from recipient 700 by pulling passer tube 104 out through second opening 708 (or first opening 704), as indicated by arrow 1102. After passer tube 104 has been removed from recipient 700, the connection of connector 1006 to second medical device 1004 (or first medical device 1002) may be completed, as shown in FIG. 12.

In some examples, first medical device 1002 implanted at first location 706 may be an electrode of an electrode lead, second medical device 1004 implanted at second location 710 may be an IPG, and connector 1006 may be a wiring portion of the electrode lead. The electrode may be positioned adjacent to or around a nerve (e.g., the pudendal nerve) at first location 706 and the proximal end and wiring portion of the electrode lead may be inserted through passer tube 104 and pushed from first location 706 to second location 710. After removal of passer tube 104 from recipient 700 through second opening 708, the proximal end of the electrode lead may be connected to the IPG. Alternatively, the electrode lead may first be connected to the IPG and then inserted through passer tube 104 and positioned (after passer tube 104 is removed) adjacent to or around the nerve.

In the IPG and electrode lead example described above, tunneling system 100 is inserted at the electrode implantation site (e.g., first location 706) and pushed toward the IPG implantation site (e.g., second location 710). However, tunneling system 100 may alternatively be used in the opposite direction by being inserted at the IPG implantation site (e.g., second location 710) and pushed toward the electrode implantation site (e.g., first location 706). Similarly, the electrode lead may be introduced into passer tube 104 from either end of passer tube 104 (e.g., from first location 706 or from second location 710).

In some examples, the IPG and electrode lead may be included in a system configured to treat detrusor sphincter dyssynergia (DSD). For example, the IPG may be implanted in the lower back along the lumbar spine and the electrode of the electrode lead may be positioned adjacent to or around the pudendal nerve. The proximal end of the electrode lead may be connected to the IPG using tunneling system 100, as described above. In some examples, passer tube 104 is large enough that the distal ends of two electrode leads may be connected to the pudendal nerve and the proximal ends of the two electrode leads may be passed through passer tube 104 to the IPG implantation site together. That is, two electrode leads may be positioned in passer tube 104 at the same time. This can obviate the need to insert tunneling system 100 through the recipient a second time to implant the second electrode lead at the nerve site, thereby reducing injury to the recipient and reducing time spent in surgery for the recipient and the surgical team.

First medical device 1002 and second medical device 1004 are not limited to an IPG and electrode lead but may be implemented by any other suitable medical devices that may be electrically, fluidically, and/or mechanically connected by connector 1006. For example, first medical device 1002 and second medical device 1004 may be a fluid reservoir (e.g., a drug reservoir), a pump, a valve, a catheter, or other device configured to hold and/or move fluids. Connector 1006 may be a catheter, tube, or other device used for holding and/or transporting fluids between first medical device 1002 and second medical device 1004.

In the examples of FIGS. 7-11, first opening 704 and second opening 708 are used to implant first medical device 1002 and second medical device 1004 at first location 706 and second location 710, respectively. In alternative examples, only one opening is used. For example, tunneling system 100 may be inserted into recipient through first opening 704 and pushed from first location 706 to second location 710. Tunneling system 100 may be guided to second location 710 in any suitable way, such as by stereotactic guidance and/or image guidance (e.g., radioscopic, ultrasonic, or fluorescence guidance). Trocar assembly 102 may then be removed from passer tube 104 and second medical device 1004 may be implanted at second location 710 by passing second medical device 1004 through passer tube 104. Second medical device 1004 may be implanted at second location 710 without the need to create or use second opening 708 because trocar assembly 102 may be removed from passer tube 104 (and passer tube 104 may be removed the recipient) in a direction opposite to the direction of insertion of tunneling system 100 into the recipient and without disconnecting piercing tip 110 from shaft 106.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A trocar assembly comprising:
an elongate shaft;
a piercing tip at a distal end of the shaft;
a handle at a proximal end of the shaft; and
a retaining member configured, when a passer tube is positioned over the shaft, to engage with an inner surface or an outer surface of the passer tube to retain a position of the passer tube over the shaft.

2. The trocar assembly of claim 1, wherein the retaining member comprises an O-ring.

3. The trocar assembly of claim 2, wherein the O-ring is positioned in a groove on the shaft and is configured to engage with the inner surface of the passer tube.

4. The trocar assembly of claim 2, wherein:
the handle comprises a hollow channel configured to receive a proximal end of the passer tube when the passer tube is positioned over the shaft; and
the O-ring is positioned in the hollow channel and is configured to engage with the outer surface of the proximal end of the passer tube.

5. The trocar assembly of claim 1, wherein the retaining member comprises a pressing member configured to press against the inner surface or the outer surface of the passer tube.

6. The trocar assembly of claim 1, wherein the retaining member comprises a seal member positioned in the handle and configured to engage with the outer surface of the passer tube.

7. The trocar assembly of claim 6, wherein the seal member is formed integrally with the handle.

8. The trocar assembly of claim 6, wherein the handle is overmolded on the seal member.

9. The trocar assembly of claim 1, wherein the retaining member is positioned on the shaft.

10. The trocar assembly of claim 1, wherein the retaining member is positioned in the handle.

11. The trocar assembly of claim 1, wherein the retaining member is configured to retain the position of the passer tube over the shaft by friction.

12. The trocar assembly of claim 1, further comprising an additional retaining member configured, when the passer tube is positioned over the shaft, to engage with the inner surface or the outer surface of the passer tube to further retain the position of the passer tube over the shaft.

13. The trocar assembly of claim 1, wherein the piercing tip is formed integrally with the shaft.

14. The trocar assembly of claim 1, wherein a maximum outer diameter of the piercing tip is less than an inner diameter of the passer tube.

15. The trocar assembly of claim 1, wherein the passer tube is removable from the shaft by application of a removing force to the trocar assembly that exceeds a retaining force applied by the retaining member to retain the passer tube over the shaft.

16. A method comprising:
inserting a trocar assembly into a proximal end of a passer tube, the trocar assembly comprising a shaft and a piercing tip at a distal end of the shaft;
pushing the trocar assembly through the passer tube so that the piercing tip emerges from a distal end of the passer tube and the passer tube is positioned over the shaft;
pushing, while the passer tube is positioned over the shaft, the piercing tip from a first location within a recipient to a second location within the recipient; and
removing the trocar assembly from the passer tube, the removing comprising pulling the trocar assembly back through the passer tube so that the shaft and the piercing tip emerge from the proximal end of the passer tube.

17. The method of claim 16, further comprising:
implanting an electrode of an electrode lead at the first location; and
passing a proximal end of the electrode lead through the passer tube from the first location to the second location.

18. The method of claim 17, wherein the implanting comprises connecting the electrode to the pudendal nerve.

19. The method of claim 17, further comprising:
implanting an implantable pulse generator (IPG) at the first location; and
connecting the proximal end of the electrode lead to the IPG.

20. The method of claim 17, further comprising:
implanting an additional electrode of an additional electrode lead at the first location; and
passing a proximal end of the additional electrode lead through the passer tube to the second location.

21. The method of claim 17, further comprising removing the passer tube from the recipient.

22. The method of claim 16, further comprising:
passing an electrode of an electrode lead through the passer tube from the second location to the first location; and
implanting the electrode at the first location.

23. The method of claim 16, wherein the piercing tip is pushed from the first location to the second location under stereotactic guidance or image guidance.

24. The method of claim 16, wherein the trocar assembly includes a retaining member that, when the passer tube is positioned over the shaft, engages with an inner surface or an outer surface of the passer tube to retain a position of the passer tube over the shaft.

25. The method of claim 24, wherein the removing the trocar assembly from the passer tube comprises pulling the trocar assembly with a removing force that exceeds a retaining force applied by the retaining member to retain the passer tube over the shaft.

26. The method of claim 25, wherein the retaining force comprises friction.

27. The method of claim 24, wherein the retaining member comprises an O-ring.

28. The method of claim 27, wherein the O-ring is positioned in a groove on the shaft and is configured to engage with the inner surface of the passer tube.

29. The method of claim 27, wherein:
the trocar assembly further includes a handle comprising a hollow channel configured to receive a proximal end of the passer tube when the passer tube is positioned over the shaft; and
the O-ring is positioned in the hollow channel and is configured to engage with the outer surface of the proximal end of the passer tube.

30. The method of claim 24, wherein the retaining member comprises a pressing member configured to press against the inner surface or the outer surface of the passer tube.

31. The method of claim 24, wherein the retaining member comprises a seal member configured to engage with the outer surface of the passer tube.

32. The method of claim 31, wherein:
the trocar assembly further includes a handle comprising a hollow channel configured to receive a proximal end of the passer tube when the passer tube is positioned over the shaft; and
the seal member is formed integrally with the handle.

33. The method of claim 31, wherein:
the trocar assembly further includes a handle comprising a hollow channel configured to receive a proximal end of the passer tube when the passer tube is positioned over the shaft; and
the handle is overmolded on the seal member.

34. The method of claim 24, wherein the retaining member is positioned on the shaft.

35. The method of claim 24, wherein the retaining member is positioned in a handle of the trocar assembly.

36. The method of claim 24, wherein the trocar assembly further comprises an additional retaining member that, when the passer tube is positioned over the shaft, engages with the inner surface or the outer surface of the passer tube to further retain the passer tube over the shaft.

37. The method of claim 16, wherein the piercing tip is formed integrally with the shaft.

38. The method of claim 16, wherein a maximum outer diameter of the piercing tip is less than an inner diameter of the passer tube.

39. A tunneling system comprising:
a passer tube; and
a trocar assembly comprising:
an elongate shaft;
a piercing tip at a distal end of the shaft;
a handle at a proximal end of the shaft; and
a retaining member that, when the passer tube is positioned over the shaft, engages with an inner surface or an outer surface of the passer tube to retain the passer tube over the shaft.

40. The tunneling system of claim 39, wherein the retaining member comprises an O-ring positioned in a groove on the shaft and configured to engage with the inner surface of the passer tube.

41. The tunneling system of claim 39, wherein:
the handle comprises a hollow channel configured to receive a proximal end of the passer tube when the passer tube is positioned over the shaft; and
the retaining member comprises an O-ring positioned in the hollow channel and configured to engage with the outer surface of the proximal end of the passer tube.

42. The tunneling system of claim 39, wherein the retaining member is configured to retain the position of the passer tube over the shaft by friction.

43. The tunneling system of claim 39, further comprising an additional retaining member configured, when the passer tube is positioned over the shaft, to engage with the inner surface or the outer surface of the passer tube to further retain the position of the passer tube over the shaft.

44. The tunneling system of claim 39, wherein the piercing tip is formed integrally with the shaft.

45. The tunneling system of claim 39, wherein a maximum outer diameter of the piercing tip is less than an inner diameter of the passer tube.

46. The tunneling system of claim 39, wherein the passer tube is removable from the shaft by application of a removing force to the trocar assembly that exceeds a retaining force applied by the retaining member to retain the passer tube over the shaft.
